(19) United States
(12) Patent Application Publication
(10) Pub. No.: US 2015/0160228 A1
(43) Pub. Date: Jun. 11, 2015

(54) COMPOSITIONS AND METHOD FOR DETERMINING THE EFFICACY OF AMYLOIDOSIS TREATMENTS

(71) Applicant: Susan CATALANO, Pittsburgh, PA (US)

(72) Inventor: Susan CATALANO, Pittsburgh, PA (US)

(21) Appl. No.: 13/021,872

(22) Filed: Feb. 7, 2011

Related U.S. Application Data
(60) Provisional application No. 61/301,905, filed on Feb. 5, 2010.
(60) Provisional application No. 61/309,094, filed on Mar. 1, 2010.

(51) Int. Cl. G01N 33/68; C07K 1/14; C07K 1/02; C07K 14/435

(52) U.S. Cl. CPC G01N 33/68; G01N 2500/20; G01N 2500/02

(57) ABSTRACT

Embodiments described herein are directed to a composition for preparing amyloid β fibrils or oligomers in vitro, testing compounds with putative amyloid β activity, amyloid β fibrils and/or oligomers prepared using such compositions, and proteins identified as being associated with amyloid β fibrils and oligomers in the composition.

### CROSS-REFERENCE TO RELATED APPLICATIONS

This application claims priority to U.S. Provisional Application No. 61/301,905, filed Feb. 5, 2010, and U.S. Provisional Application No. 61/309,094, filed Mar. 1, 2010, each of which is incorporated in its entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

Not applicable

### BRIEF SUMMARY OF THE INVENTION

Embodiments described herein include a composition for preparing protein complexes including neural proteins suspended in an aqueous solution to form a neural protein slurry and amyloid β admixed into the neural protein slurry. In some embodiments, the neural proteins may be brain proteins, and in other embodiments, the neural proteins may be derived from the brain of a mammal such as but not limited to rat, mouse, dog, monkey, ape, or human. In various embodiments, the amyloid β may be amyloid β monomers, amyloid oligomers of about 2 to about 200 amyloid β monomers, or combinations thereof, and in some embodiments, the amyloid β may be amyloid β monomers, amyloid β oligomers of 2 or 100 amyloid β monomers, and combinations thereof.

The protein complexes of such embodiments may include amyloid β oligomers associated with one or more neural proteins from the neural protein slurry, and in some embodiments, the protein complexes may include amyloid β fibrils associated with one or more neural proteins from the neural protein slurry. In certain embodiments, the aqueous solution may include one or more additional components selected such as, but not limited to, protease inhibitors, buffers, chelating agents, metals, metal salts, and combinations thereof, and in particular embodiments, the aqueous solution may include metal ions such as, but not limited to Cu(ii), Zn(II), Fe(II), Fe(III), and Mg(II).

Further embodiments are directed to methods for assembling amyloid β including the steps of combining neural proteins in an aqueous solution to form a neural protein slurry, admixing amyloid β into the neural protein slurry to form a neural protein/amyloid β mixture, and assembling protein complexes in the neural protein/amyloid β mixture. In some embodiments, the neural proteins may be brain proteins, and in other embodiments, the neural proteins may be derived from the brain of a mammal such as but not limited to rat, mouse, dog, monkey, ape, or human. In various embodiments, the amyloid β may be amyloid β monomers, amyloid β oligomers of about 2 to about 200 amyloid β monomers, or combinations thereof, and in some embodiments, the amyloid β may be amyloid β monomers, amyloid β oligomers of 2 or 100 amyloid β monomers, and combinations thereof. In certain embodiments, the aqueous solution may include one or more additional components selected such as, but not limited to, protease inhibitors, buffers, chelating agents, metals, metal salts, and combinations thereof, and in particular embodiments, the aqueous solution may include metal ions such as, but not limited to Cu(ii), Zn(II), Fe(II), Fe(III), and Mg(II). In some embodiments, the amyloid β oligomers may include one or more neural proteins from the neural protein slurry. In other embodiments, the protein complexes may be amyloid β fibrils, and in still other embodiments, the protein complexes may be amyloid β fibrils associated with one or more neural proteins.

In certain embodiments, the method may include the step of isolating the protein complexes, and in some embodiments, the method may further include the step of isolating one or more neural proteins that associate with amyloid β. In other embodiments, such methods may include the step of identifying the one or more neural proteins that associate with amyloid β.

In further embodiments, the method may include the step of adding one or more compositions having putative anti-amyloid β activity to the neural protein/amyloid β mixture. In some embodiments, such methods the composition may be added after assembling protein complex, and in other embodiments, the composition may be added before assembling protein complexes. The composition of such embodiments may be any type of composition known in the art such as, but not limited to, one or more small molecule, protein, peptide, or combinations thereof. In particular embodiments, the method may further include the step of identifying compounds that inhibit, reduce, or reverse assembly of the protein complexes, and in some embodiments, the identified compounds may be used to treat amyloidosis.

Further embodiments include a protein complex prepared by a method including the steps of combining neural proteins in an aqueous solution to form a neural protein slurry, admixing amyloid β into the neural protein slurry to form a neural protein/amyloid β mixture, assembling protein complexes from the neural protein/amyloid β mixture, and isolating the protein complexes. In some embodiments, the protein complexes may include one or more neural proteins associated with the amyloid β. In other embodiments, the protein complexes may include amyloid β monomers associated with one or more neural proteins. In still other embodiments, the protein complexes may include amyloid β oligomers associated with one or more neural proteins, and in yet further embodiments, the protein complexes may include amyloid β fibrils associated with one or more neural proteins.

Still further embodiments include methods for identifying a compound that inhibits amyloid β assembly including the steps of combining neural proteins in an aqueous solution to form a neural protein slurry, admixing amyloid β into the neural protein slurry to form a neural protein/amyloid β mixture, adding one or more compositions having putative anti-amyloid β activity to the neural protein/amyloid β mixture, and identifying compounds that inhibit or reduce amyloid β assembly.

Yet other embodiments include compounds identified by a method including the steps of combining neural proteins in an aqueous solution to form a neural protein slurry;, admixing amyloid β into the neural protein slurry to form a neural protein/amyloid β mixture, adding one or more compositions having putative anti-amyloid β activity to the neural protein/amyloid β mixture, and identifying compounds that inhibit or reduce amyloid β assembly.

Still other embodiments include methods for identifying a compound that degrade amyloid β assemblies including the steps of combining neural proteins in an aqueous solution to form a neural protein slurry, admixing amyloid β into the neural protein slurry to form a neural protein/amyloid β mixture, assembling amyloid β assemblies from the amyloid β, adding one or more compositions having putative anti-amyloid β activity to the neural protein/amyloid mixture, and identifying compounds that degrade amyloid β or inhibit further amyloid β assembly.

Certain embodiments are directed to compounds identified by a method including the steps of combining neural proteins in an aqueous solution to form a neural protein slurry, admixing amyloid β into the neural protein slurry to form a neural protein/amyloid β mixture, assembling amyloid β fibrils from the amyloid β, adding one or more compositions having putative anti-amyloid β activity to the neural protein/amyloid β mixture, and identifying compounds that degrade amyloid β or inhibit further amyloid β assembly.

### DESCRIPTION OF DRAWINGS

FIG. 1 shows mass spectroscopy of an amyloid β containing solution.

FIG. 2 shows mass spectroscopy of an amyloid β containing solution after treatment with tri-fluoro ethanol.

### DETAILED DESCRIPTION

Before the compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth.

As used herein, the term “about” means plus or minus 10% of the numerical value of the number with which it is being used. Therefore, about 50% means in the range of 45%-55%.

“Administering” when used in conjunction with a therapeutic means to administer a therapeutic directly into or onto a target tissue or to administer a therapeutic to a patient, whereby the therapeutic positively impacts the tissue to which it is targeted. Thus, as used herein, the term “administering”, when used in conjunction with a nitrated lipid, can include, but is not limited to, providing a nitrated lipid to a subject systemically by, for example, intravenous injection, whereby the therapeutic reaches the target tissue. “Administering” a composition may be accomplished by, for example, injection, oral administration, topical administration, or by these methods in combination with other known techniques. Such combination techniques include heating, radiation, ultrasound and the use of delivery agents.

The term “animal” as used herein includes, but is not limited to, humans and non-human vertebrates such as wild, domestic and farm animals.

The term “improves” is used to convey that the present invention changes either the characteristics and/or the physical attributes of the tissue to which it is being provided, applied or administered. The term “improves” may also be used in conjunction with a diseased state such that when a diseased state is “improved” the symptoms or physical characteristics associated with the diseased state are diminished, reduced or eliminated.

The term “inhibiting” includes the administration of a compound of the present invention to prevent the onset of the symptoms, alleviating the symptoms, or eliminating the disease, condition or disorder.

By “pharmaceutically acceptable”, it is meant the carrier, diluent or excipient must be compatible with the other ingredients of the formulation and not deleterious to the recipient thereof.

As used herein, the term “therapeutic” means an agent utilized to treat, combat, ameliorate, prevent or improve an unwanted condition or disease of a patient. In part, embodiments of the present invention are directed to the treatment of inflammation, obesity-related diseases, metabolic diseases, cardiovascular diseases, cerebrovascular and neurodegenerative diseases, cancer or the aberrant proliferation of cells.

A “therapeutically effective amount” or “effective amount” of a composition is a predetermined amount calculated to achieve the desired effect, i.e., to inhibit, block, or reverse the activation, migration or proliferation of cells. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. However, it will be understood that the effective amount administered will be determined by the physician in the light of the relevant circumstances including the condition to be treated, the choice of compound to be administered, and the chosen route of administration; and therefore, the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The terms “treat”, “treated”, or “treating” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

Generally speaking, the term “tissue” refers to any aggregation of similarly specialized cells which are united in the performance of a particular function.

Human amyloid β is the cleavage product of an integral membrane protein, amyloid precursor protein (APP), found concentrated in the synapses of neurons. Amyloid β self-associates to form metastable, globular oligomeric assemblies that at higher concentrations under lower pH conditions, polymerize and assemble into fibrils. Amyloid β oligomers have been demonstrated to cause Alzheimer's disease by inducing changes in neuronal synapses that block learning and memory, and amyloid β fibrils have long been associated with the advanced stages Alzheimer's disease. However, very little is known about the intracellular or extracellular location of oligomer formation and the structural state of the oligomer. For example, the number of amyloid β subunits that associate to form the oligomer is currently unknown, as is the structural form of the oligomers, which residues are exposed, and whether more than one structural state of oligomer is neuroactive.

Amyloid β has affinity for many proteins found in the brain, including ApoE and ApoJ. However, it is unclear whether chaperones or other proteins form associations with the protein that can affect its final structural state and/or its neuroactivity.

Embodiments of the invention are generally directed to compositions at least including proteins, such as neural proteins, suspended in an aqueous solution and monomers or oligomers of amyloid β. The compositions embodied herein allow in vitro formation of protein complexes and amyloid β assemblies formed in a medium that reflects the environment of cells in which such protein complexes and amyloid β assemblies would form during progression of certain amyloidosis related diseases such as, for example, Alzheimer's disease, in vivo. Thus, the compositions of various embodiments provide an in vitro system for preparing amyloid β assemblies that may include amyloid β oligomers and/or amyloid β fibrils as well as other neural proteins that associate with amyloid β monomers, amyloid β oligomers and/or amyloid β fibrils. Other embodiments are directed to methods for identifying proteins that associate with amyloid β monomers, amyloid β oligomers and/or amyloid β fibrils, and testing the efficacy of compounds designed to inhibit, reduce, or degrade amyloid β assemblies and/or amyloid β oligomer and/or amyloid β fibril. Yet other embodiments are directed to methods for using such compositions in, for example, preparing various amyloid β assemblies such as amyloid β oligomers and/or amyloid β fibrils and compositions that include amyloid β oligomers and/or amyloid β fibrils prepared using such compositions and proteins and compounds identified using the methods of various embodiments.

As used herein, the term “protein complex” or “protein complexes” may be used to refer two or more proteins that form an association in the aqueous solution of proteins and amyloid β embodied by the invention. In some embodiments, the protein complexes may include amyloid β monomers and/or amyloid β oligomers and proteins from the aqueous solution, and protein complexes that include amyloid β monomers, amyloid β oligomers, and/or amyloid β fibrils associated with one or more proteins from the aqueous solution of proteins may be referred to as “amyloid β assemblies.” In other embodiments, the protein complexes may include no amyloid β. Without wishing to be bound by theory, such non-amyloid β containing complexes may be indicative of protein complexes that form during the assembly of amyloid β oligomers and/or amyloid β fibrils but that dissociate from amyloid β oligomers and/or amyloid fibrils after assembly such as, for example, chaperone complexes. Alternatively, protein complexes may form in the presence of amyloid β as a result of, for example, an intracellular signaling cascade. Protein complexes are not limited by the type of association formed between the two or more proteins. For example, in some embodiments, the association may be a non-covalent interaction such as, for example, ionic bonds between the protein components, and in other embodiments, the two or more proteins may be associated by a covalent interaction such as, for example, a disulfide bond. In still other embodiments, the association between the two or more proteins may be a combination of non-covalent and covalent interactions, and in some such embodiments, a covalent interaction may be induced during formation in order, for example, to trap the two or more proteins in the association.

The compositions of embodiments at least include proteins suspended in an aqueous solution and amyloid β monomers and/or amyloid oligomers. The proteins of various embodiments may be obtained from any source, and in particular embodiments the proteins may be obtained from a mammal such as, but not limited to, a mouse, rat, ape, monkey, dog, cat, or human. In some embodiments, the proteins may be neural proteins, and in such embodiments the neural proteins may be obtained form nervous tissue or brain tissue. For example, in some embodiments, neural proteins may be obtained from a brain extract or slurry prepared by pulverizing brain tissue, and in other embodiments neural proteins may be obtained from specific types of brain or nervous tissue by first dissecting the specific type of tissue away from other nervous tissue and blending or pulverizing the specific tissue. In other embodiments, the proteins may be non-neural proteins obtained from tissues other than nervous or brain tissues. For example, amyloidosis is known to effect kidneys, skin, digestive tract, lymph nodes, blood vessels, blood, and various organs associated with these; and in some embodiments, proteins used in the compositions may be obtained from these tissues.

The aqueous solution of the composition may include any number of additional components including, for example, one or more compounds or compositions for adjusting pH of the compositions, one or more compounds or compositions for adjusting salt concentrations, one or more compounds or compositions for stabilizing the protein components of the composition such as, but not limited to, protease inhibitors, buffers, chelating agents, metals, metal salts, and the like. Such additional components are well known in the art, and any known components may be used in various embodiments. For example, in some embodiments, the pH of the composition may be from about 6.5 to about 8.0 or about 7.0 to about 7.5, and any buffer, acid, or base may be added to the composition to achieve the appropriate pH. In other embodiments, the salt concentration of the composition may be adjusted to appropriate concentrations by adding sodium chloride such that the final concentration is about 0.8% to about 10% or about 0.9%. In still other embodiments, the metal ions may be provided in the mixture by including one or more metals or metal salts including, for example, Cu(II), Zn(II), Fe (II), Fe(III), Mg(II), and the like. Without wishing to be bound by theory, the inclusion of metal ions in the solution may enhance oligomerization and/or stabilize amyloid β oligomers.

The monomers or oligomers of amyloid β may be obtained from any source. For example, in some embodiments, commercially available amyloid β monomers and/or amyloid β oligomers may be used in the aqueous solution, and in other embodiments, amyloid β monomers and/or amyloid β oligomers that are used in the aqueous protein solution can be isolated and purified by the skilled artisan using any number of known techniques. In general, the amyloid β monomers and/or amyloid β oligomers used in the preparation of the aqueous solution of proteins and amyloid β of various embodiments may be soluble in the aqueous solution. Therefore, both the proteins of the aqueous solution and the amyloid β may be soluble.

The amyloid β added may be of any isoform. For example, in some embodiments, the amyloid β monomers may be amyloid β 1-42, and in other embodiments the amyloid β monomers may be amyloid β 1-40 or. In still other embodiments, the amyloid β may be amyloid β 1-39 or amyloid β 1-41. Hence, the amyloid β of various embodiments may encompass any C-terminal isoform of amyloid β. Yet other embodiments include amyloid β in which the N-terminus has been frayed, and in some embodiments, the N-terminus of any of amyloid β C-terminal isomers described above may be amino acid 2, 3, 4, 5, or 6. For example, amyloid β 1-42 may encompass amyloid β 2-42, amyloid β 3-42, amyloid β 4-42, or amyloid 5-42 and mixtures thereof, and similarly, amyloid β 1-40 may encompass amyloid β 2-40, amyloid β 3-40, amyloid β 4-40, or amyloid β 5-40.

The amyloid β used in various embodiments may be wild type, i.e. having an amino acid sequence that is identical to the amino acid sequence of amyloid β synthesized in vivo by the majority of the population, or in some embodiments, the amyloid β may be a mutant amyloid β. Embodiments are not limited to any particular variety of mutant amyloid β. For example, in some embodiments, the amyloid β introduced into the aqueous solution may include a known mutation, such as, for example, amyloid β having the “Dutch” (E22Q) mutation or the “Arctic” (E22G) mutation. Such mutated monomers may include naturally occurring mutations such as, for example, forms of amyloid β isolated from populations of individuals that are predisposed to, for example, Alzheimer's disease, familial forms of amyloid β. In other embodiments, mutant amyloid β monomers may be synthetically produced by using molecular techniques to produce an amyloid β mutant with a specific mutation. In still other embodiments, mutant amyloid β monomers may include previously unidentified mutations such as, for example, those mutants found in randomly generated amyloid β mutants. The term “amyloid β” as used herein is meant to encompass both wild type forms of amyloid β as well as any of the mutant forms of amyloid β.

In some embodiments, the amyloid β in the aqueous protein solution may be of a single isoform. In other embodiments, various C-terminal isoforms of amyloid β and/or various N-terminal isoforms of amyloid β may be combined to form amyloid β mixtures that can be provided in the aqueous protein solution. In yet other embodiments, the amyloid β may be derived from amyloid precursor protein (APP) that is added to the protein containing aqueous solution and is cleaved in situ, and such embodiments, various isoforms of amyloid β may be contained within the solution. Fraying of the N-terminus and/or removal of C-terminal amino acids may occur within the aqueous solution after amyloid β has been added. Therefore, aqueous solutions prepared as described herein may include a variety of amyloid β isoforms even when a single isoform is initially added to the solution.

The amyloid β used in various embodiments may be derived from any source. For example, in some embodiments, the amyloid β monomers added to the aqueous solution may be isolated from a natural source such as living tissue, and in other embodiments, the amyloid β may be derived from a synthetic source such as transgenic mice or cultured cells. In certain embodiments, amyloid β monomers or amyloid β oligomers may derived from a source expressing a single form of amyloid β, and in other embodiments, a combination of amyloid β monomers and amyloid β oligomers may be expressed in a single source to produce ologimers that contain more than one form of amyloid β. In still other embodiments, amyloid β monomers and amyloid β oligomers may be derived from different sources, and in such embodiments, the individual sources may produce the same or different forms of amyloid β monomer. It is noted that various isoforms of each type or form of amyloid β monomer may be included in every collection of amyloid β monomers, as N-terminal and C-terminal fraying may occur before the amyloid β is added to the aqueous solution regardless of the source or form of amyloid β monomer used. Additionally, amyloid β oligomers used in preparing the aqueous solution and/or the oligomers that are assembled in the solution may be homo-oligomers, which contain a single isoform of amyloid β, or hetero-oligomers, which contain more than one isoform of amyloid β.

In some embodiments, the amyloid β monomers, oligomers, or combinations thereof are isolated from normal and/or patients that have been diagnosed with cognitive decline or diseases associated therewith, such as, but not limited to, Alzheimer's disease. In some embodiments, the amyloid β monomers, oligomers, or combinations thereof are Abeta assemblies that have been isolated from normal or diseased patients. In some embodiments, the Abeta assemblies are high molecular weight, e.g. greater than 100 KDa. In some embodiments, the Abeta assemblies are intermediate molecular weight, e.g. 10 to 100 KDa. In some embodiments, the Abeta assemblies are less than 10 KDa.

In particular embodiments, methods described herein may include the step of determining the effect of mutant amyloid β on oligomer and/or fibril/protofibril formation using in vitro assays that include introducing mutant amyloid β into the aqueous solution. In some such embodiments, methods may include, for example, the steps of adding mutant amyloid β to the aqueous solution and observing oligomerization and/or fibril/protofibril assembly of the mutant amyloid β or determining the rate or extent of oligomerization and/or fibril/protofibril assembly.

The amyloid β oligomers of embodiments may be composed of any number of amyloid β monomers consistent with the commonly used definition of “oligomer.” For example, in some embodiments, amyloid β oligomers may include from 2 to about 200 amyloid β monomers, and in other embodiments, amyloid β oligomers may be composed from about 2 to about 150, about 2 to about 100, about 2 to about 50, or about 2 to about 25, amyloid β monomers. The amyloid β oligomers of various embodiments may be distinguished from amyloid β fibrils and amyloid β protofibrils based on the confirmation of the monomers. In particular, the amyloid β monomers of amyloid β oligomers are generally globular consisting of β-pleated sheets whereas secondary structure of the amyloid β monomers of fibrils and protofibrils is parallel β -sheets.

The concentration of amyloid β monomer and/or oligomer in the composition may vary among embodiments and may vary depending on the use of the composition. For example in some embodiments, about 0.1% to about 75%, about 0.5% to about 65% or about 1% to about 50% of the protein components of the composition may be amyloid β monomer or amyloid β oligomer or a combination of amyloid β monomer and oligomer. In other embodiments, the concentration of amyloid β monomer and/or oligomer may be from about 1 pM to about 25 mM, about 50 pM to 20 mM, about 1 nM to about 15 mM, or about 0.5 μM to about 10 mM.

Without wishing to be bound by theory, amyloid β monomers and/or oligomers may begin self-assembly into larger globular oligomers spontaneously upon being added to the protein containing aqueous solution. Therefore, any composition embodied by the invention may include a combination of monomers, oligomers having various numbers of subunits. The composition of some embodiments may further include amyloid protofibrils and amyloid β fibrils which spontaneously form in the aqueous solution or that were added as part of the amyloid β added to the solution. In various embodiments, the concentration of amyloid β monomers and oligomers, and/or in some cases, protofibrils and fibrils, in the composition may vary from the concentration of these elements in the constituent parts and throughout the lifetime of the composition. These species may be in dynamic equilibrium with one another as oligomers, protofibrils, and fibrils may dissociate into monomers which can than reform as new species of oligomers, protofibrils, and fibrils.

Further embodiments are directed to the method for using such compositions. For example, in some embodiments, compositions including proteins suspended in an aqueous solution amyloid β monomers may be used to produce protein complexes that can include amyloid β oligomers, and in other embodiments, the protein complexes may include amyloid β monomers amyloid β oligomers and/or amyloid β fibrils. In such embodiments, the method may include the steps of combining proteins in an aqueous solution, admixing into this solution amyloid β monomers and/or oligomers, and allowing protein complexes that include amyloid monomers, amyloid oligomers and/or amyloid β fibrils to assemble in the solution. In some embodiments, such protein complexes may form immediately upon the addition of amyloid β monomers and/or amyloid β oligomers to the protein solution. In other embodiments, assembling such protein complexes may be carried out for a period of time to allow the amyloid β oligomers and/or amyloid β fibrils to achieve an appropriate size. For example, in some embodiments, assembling protein complexes may be carried out for about 1 minute to about 48 hours. In other embodiments, the period of time may vary to produce protein complexes including amyloid β oligomers and/or amyloid β fibrils of a specific size. For example, larger amyloid β oligomers or amyloid β fibrils may be produced by allowing assembly to occur for 24 or more hours up to about 7 days, 2 weeks, or 1 or more months, and smaller amyloid β oligomers or amyloid β fibrils may be produced by allowing assembly to occur for several minutes to 1 or 2 hours up to 1 or more days or 1 or more weeks. In addition, the amyloid β oligomers or amyloid β fibrils and/or amyloid assemblies formed during the time period may be sufficiently stable that the amyloid β oligomers or amyloid β fibrils and/or amyloid assemblies formed may be stored in the aqueous protein solution for from 1 or more days up to 1 to 6 months.

In some embodiments, such method may further include isolating and/or purifying the protein complexes formed in the solution after assembling the protein complexes. The step of isolating and/or purifying the protein complexes from the solution may be carried out by any method, and in some embodiments, the methods for isolating and purifying protein complexes may be similar to or encompass one or more steps for isolating or purifying amyloid β oligomers or amyloid β fibrils known in the art. For example, in some embodiments, methods for isolating and/or purifying protein complexes may include sequential filtration, centrifugation, pellet homogenization/resuspension, and further centrifugation steps, and in such embodiments, amyloid β oligomers and/or amyloid β fibril may be contained within pellets which may be are resuspended in buffers designed to remove additional protein components mixed with the amyloid β fibrils in the pellets. Such buffers may include sodium dodecyl sulfate (SDS), which may denature or partially denature cellular proteins while amyloid β oligomers and/or amyloid β fibrils remain intact, and in some embodiments, methods for isolating and/or purifying protein complexes may include one or more heating steps. In other embodiments, protein complexes that include amyloid β monomers, amyloid β oligomers, and/or amyloid β fibrils may be isolated and/or purified by immunoprecipitation, using antibodies that recognize one or more isoform of amyloid β. Numerous antibodies useful for immunoprecipitation of amyloid β are known in the art, and any such antibodies may be used in embodiments.

In certain embodiments, the methods described above may include identifying proteins that make up the protein complexes prepared in the aqueous solution which may or may not be associated with the amyloid β oligomers or amyloid β fibrils. The step of identifying proteins in the protein complexes may be carried out either before or after isolation and/or purification of the protein complexes assembled in the protein suspension. For example in some embodiments, proteins associated with amyloid β monomers, amyloid β oligomers, and/or amyloid β fibrils may be identified by two dimensional gel electrophoresis of a sample of the solution after assembling the protein complexes. In other embodiments, amyloid β monomers, amyloid β oligomers, and/or amyloid β fibrils may be isolated and/or purified or partially isolated and/or purified before identifying proteins in the protein complex, and in such embodiments, these proteins may be associated with the amyloid β monomers, amyloid oligomers, and/or amyloid β fibrils or separate protein complexes that associate with amyloid β monomers, amyloid β oligomers, and/or amyloid β fibrils. For example, in some embodiments, SDS-PAGE or mass spectroscopy analysis may be performed on isolated and/or purified or partially isolated and/or purified protein complexes. In other embodiments, the isolated or purified or partially isolated or partial purified protein complexes may be further treated degrade or denature additional protein components that are not associated with amyloid β monomers, amyloid β oligomers, and/or amyloid β fibrils in the protein complexes. For example, in some embodiments, isolated or purified or partially isolated or partially purified amyloid β monomers, amyloid β oligomers, and/or amyloid β fibrils may be treated with trifluoroethanol (TFE). Without wishing to be bound by theory, assembled structures and none-covalently bound protein complexes may dissociate into denatured proteins in TFE, and can be removed from the solution. Therefore when, for example, mass spectroscopy is carried out on the proteins remaining in the solution, peaks associated with proteins not associated with amyloid β monomers, amyloid β oligomers, and/or amyloid β fibrils or that are only loosely associated with amyloid β monomers, amyloid β oligomers, and/or amyloid β fibrils, disappear leaving only peaks associated with proteins that are tightly associated, or covalently attached, to the amyloid β monomers, amyloid oligomers and/or amyloid β fibrils. Proteins identified using the methods may be further characterized after identification by any method known in the art.

Other embodiments are directed to methods for using the compositions of the invention for identifying compounds that affect amyloid β monomers, amyloid β oligomers, and/or amyloid β fibrils assembly or degrade amyloid β oligomers and/or amyloid β fibrils. For example, in some embodiments, methods may include the steps of combining proteins in an aqueous solution, and adding one or more compounds having putative anti-amyloid β assembly activity to the aqueous solution. Amyloid β monomers and/or oligomers may then be admixed into the aqueous solution. After allowing amyloid β oligomers and/or amyloid β fibrils to assemble in the solution, the solution may be tested to determine whether amyloid β oligomers and/or amyloid β fibrils have formed using, for example, immunoprecipitation of amyloid β oligomers and/or amyloid β fibrils and mass spectroscopy or SDS-PAGE electrophoresis. Compounds that reduce or eliminate amyloid β oligomer and/or amyloid β fibril formation, or diminish the size of amyloid β oligomers and/or amyloid β fibrils, in the solution as compared to amyloid β oligomers and/or amyloid β fibrils formed in the absence of the test compound may be further characterized for their anti-amyloid β activity as potential treatments for amyloidosis.

Methods of mass spectrometry are known and any suitable method can be used in conjunction with the methods described herein. For example, Gelfanova et al. (Quantitative analysis of amyloid-b peptides in cerebrospinal fluid using immunoprecipitation and MALDI-Tof mass spectrometry. Briefings in Funct. Genomics Proteomics. 2007, 6:149-158); Kumar et al. (Dense-core senile plaques in the Flemish variant of Alzheimer's disease are vasocentric. AmJ Pathol 2002; 161:507-20.); Wang et al. (The profile of soluble amyloid beta protein in cultured cell media. Detection and quantification of amyloid protein and variants by immunoprecipitation-mass spectrometry. J Biol Chem 1996; 271:31894-902.); and Portelius et al. (Determination of b-amyloid peptide signatures in cerebrospinal fluid using immunoprecipitation-mass spectrometry. J Proteome Res 2006, Published on Web 3 Oct. 2006, 10.1021/pr050475v.) describe various methods of using mass spectrometry to analyze amyloid beta peptides and other proteins isolated from cerebrospinal fluid or neurological tissues, each of which is hereby incorporated by reference in its entirety. Mass spectrometry can also be used as described herein and, for example, in the Examples section contained herein.

In some embodiments, mass spectrometry is used to identify agents that disrupt or inhibit protein complexes found specifically or substantially specifically in the patients having a neurological disease or cognitive decline, such as but not limited to those described herein. In some embodiments, mass spectrometry is used to identify agents that enhance the stability or cause of the formation of protein complexes found specifically or substantially specifically in the patients having a neurological disease or cognitive decline, such as but not limited to those described herein. For example, if a test compound is added to a composition described herein and a protein complex is identified as being disrupted by, for example, using mass spectrometry, then the test compound is said to be able to inhibit or disrupt the formation of a protein complex.

Mass spectrometry can also be used to isolate and identifying Abeta species or complexes in an animal brain, including but not limited to a human brain, with MALDI-Tof-MS. Abeta species that are non-covalently bound in a complex with other proteins in the human Alzheimer's disease brain (AD) can be identified using mass spectrometry. Accordingly, in some embodiments, the present invention provides methods of identifying protein complexes comprising one or more Abeta species using mass spectrometry. In some embodiments, the mass spectrometry is MALDI-Tof-MS or another method described herein. The methods can also be used to identify protein complexes that are found specifically in diseases brains and not found in, for example, age-matched non-demented human control brains. The non-demented human control brains can sometimes be referred to as normal brains.

The compositions analyzed can be a composition that is artificially created as described herein or be solely isolated from a human brain that has not had exogenous amyloid monomers or oligomers added to it. For example, a brain (e.g. mammal, primate, feline, canine, porcine, human, and the like) tissue (e.g. pre or post-mortem brain tissue) can be homogenized. In some embodiments, a protease inhibitor can be added prior to, during or after homogenization. An example of a protease inhibitor includes, but is not limited to complete protease inhibitor. The tissue homogenates can then be clarified so that the solids are cleared from the solution. This can be done, for example by centrifugation or other mechanical methods, including but not limited to ultracentrifugation. These methods can be used to isolate or purify the soluble fractions. The soluble fractions can also be immunodepleted using various methods, including but not limited to, using protein-A and/or protein-G agarose columns and the like. The purified fractions can also be size fractionated. In some embodiments, the soluble extracts are concentrated. The fractions can then be immunopreciptated with an antibody or other molecule that recognizes amyloid monomers or oligomers or amyloid precursor protein (APP). An example includes, but is not limited to an antibody referred to as 6E-10. The antibody can, in some embodiments, be conjugated to a bead (e.g. agarose). These methods can be used to isolate Abeta containing protein species from brain homogenates. These methods can also be combined or adapted to be used with any other composition and/or method described herein.

In other exemplary embodiments, methods may include the steps of combining proteins in an aqueous solution, admixing amyloid β monomers or amyloid β oligomers into the aqueous solution, allowing amyloid β fibrils and/or larger amyloid β oligomers to assemble in the solution, and adding one or more compounds having putative anti-amyloid β activity to the aqueous solution. The solution may then be tested to determine whether the state of amyloid β monomers, amyloid β oligomers, and/or amyloid β fibrils in the solution following treatment with the one or more test compound. For example, amyloid β monomers, amyloid β oligomers, and/or amyloid β fibrils may be immunoprecipitated and mass spectroscopy or SDS-PAGE electrophoresis may be used to determine the presence and/or size of the amyloid β oligomers and/or amyloid β fibrils remaining in the solution. In such embodiments, compounds that are found to eliminate amyloid β oligomers and/or amyloid β fibrils, reduce the number of amyloid oligomers and/or amyloid β fibrils, or diminish the average size of the amyloid β oligomers and/or amyloid β fibrils in the solution may be further characterized as potential treatments for amyloidosis.

Embodiments are not limited by the type or number of compounds that can be tested using the methods described above. For example, in some embodiments, the test compounds may be small molecules, and in certain embodiments, the test compounds may be small molecules that are designed to bind to amyloid β monomers, amyloid β oligomers and/or amyloid β fibrils, or proteins that associated with amyloid β monomers, amyloid β oligomers, and/or amyloid β fibrils and may reduce formation of amyloid β fibrils, inhibit growth of amyloid β oligomers and/or amyloid β fibrils, or reduce the size or eliminate preformed amyloid oligomers and/or amyloid β fibrils. In other embodiments, test compound may be a protein or other biological molecule that interacts with amyloid β monomers, amyloid β oligomers and/or amyloid β fibrils or proteins that associated with amyloid β monomers, amyloid β oligomers and/or amyloid β fibrils and may reduce formation of amyloid β oligomers and/or amyloid fibrils, inhibit growth of amyloid β oligomers and/or amyloid β fibrils, or reduce the size or eliminate preformed amyloid β oligomers and/or amyloid β fibrils. In still other embodiments, the test compound may be a combination of compounds with known anti-amyloid β activity, a combination of test compounds, or a combination of one or more compounds with known anti-amyloid β activity and one or more test compounds. In such embodiments, test compounds, combinations of test compounds, and combinations of compounds with known anti-amyloid β activity and test compounds that exhibit anti-amyloid β activity or improved anti-amyloid β activity may be further characterized for in vivo activity and further developed into anti-amyloidosis pharmaceuticals.

Embodiments are further directed to proteins that associate with amyloid identified using the methods described above, and amyloid β oligomers and amyloid β fibrils including such associated proteins. Without wishing to be bound by theory, the amyloid β oligomers and fibrils prepared by the methods of embodiments may include associated proteins that are necessarily absent from amyloid β oligomers and/or amyloid β fibrils prepared by conventional in vitro methods. Such proteins may affect, for example, stability of the amyloid β oligomers and/or amyloid β fibrils and/or the ability of test compounds or currently used treatments to inhibit amyloid β oligomers and/or amyloid β fibrils formation cause degradation of the amyloid β oligomers and/or amyloid β fibrils. For example, proteins associated with amyloid β monomers, amyloid β oligomers and/or amyloid β fibrils may act as chaperones

Thus, amyloid β monomers, amyloid β oligomers and/or amyloid β fibrils assembled in the protein containing aqueous solution of various embodiments may provide fibrils that are more reflective of amyloid β monomers, amyloid β oligomers, and/or amyloid p fibrils found in living tissue, and using such amyloid β monomers, amyloid β oligomers, and/or amyloid β fibrils to determine the effectiveness of test compounds for inhibiting amyloid β oligomers and/or amyloid β fibrils assembly or enhancing degradation of amyloid β oligomers and/or amyloid β fibrils may provide data that is more reflective of the test compounds in vivo efficacy. Additionally, preparing amyloid β oligomers and/or amyloid β fibrils in a protein containing aqueous solution may mimic fibril assembly in nervous tissue, and proteins that associate with the amyloid β monomers, amyloid β oligomers and/or amyloid β fibrils may represent proteins that are necessary for or that otherwise effect in vivo amyloid β oligomers and/or amyloid β fibrils assembly. Thus, proteins identified using the methods of embodiments described above may represent novel targets for anti-amyloidosis treatments.

The present invention also provides other methods for identifying agents or compounds or one or more compositions that can inhibit or degrade amyloid β assembly or a disease protein complex assembly. A “disease protein complex” as used herein refers to a protein complex that is found in the tissue or other biological sample (e.g. blood) from an animal (e.g. human) with a disease, but is not found in an animal without the disease. The disease can be, for example, dementia, Alzheimer's disease, Parkinson's disease, cognitive decline, and the like. The disease can also refer to an animal model that is used to mimic any disease referred to herein. Therefore, the test compound can be used to see if it inhibits a protein complex formation or amyloid complex (assembly) and as a control it can be compared to a sample that is from an animal without the disease.

In some embodiments, the present invention provides methods for identifying a compound that inhibits amyloid β assembly or a disease protein complex assembly comprising contacting one or more compositions to a composition comprising an amyloid β assembly or a disease protein complex assembly, and identifying one or more compositions as a compound that inhibits amyloid β assembly or a diseased protein complex assembly, wherein one or more compositions that inhibit amyloid β assembly or a disease protein complex assembly are identified as a one or more compositions that inhibit amyloid β assembly or a disease protein complex assembly. In some embodiments, the composition comprising an amyloid β assembly or a disease protein complex assembly is a brain homogenate. In some embodiments, the method comprises preparing the brain homogenate. Brain homogenates can be prepared by any method including those that are described herein. In some embodiments, the composition comprising an amyloid β assembly or a disease protein complex assembly is a composition comprising one or more neural proteins admixed with an amyloid β mixture. In some embodiments, the composition comprising an amyloid β assembly or a disease protein complex assembly is a neural protein slurry/amyloid β mixture. In some embodiments, the method comprises preparing the neural protein slurry/amyloid β mixture by combining a plurality of neural proteins in an aqueous solution to form a neural protein slurry; admixing amyloid β into the neural protein slurry to form a neural protein/amyloid β mixture.

As discussed throughout, the compound can be identified by using mass-spectrometry. Mass-spectrometry can be used to monitor the formation, inhibition, or degradation of the amyloid compositions or disease protein complexes.

In some embodiments, the present invention provides methods for identifying a compound that degrades amyloid β assembly or a disease protein complex assembly comprising contacting one or more compositions to a composition comprising an amyloid β assembly or a disease protein complex assembly, and identifying one or more compositions as a compound that inhibits amyloid β assembly or a diseased protein complex assembly, wherein a composition that degrades amyloid β assembly or a disease protein complex assembly is identified as a compound that degrades amyloid β assembly or a disease protein complex assembly.

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples.

### EXAMPLE 1

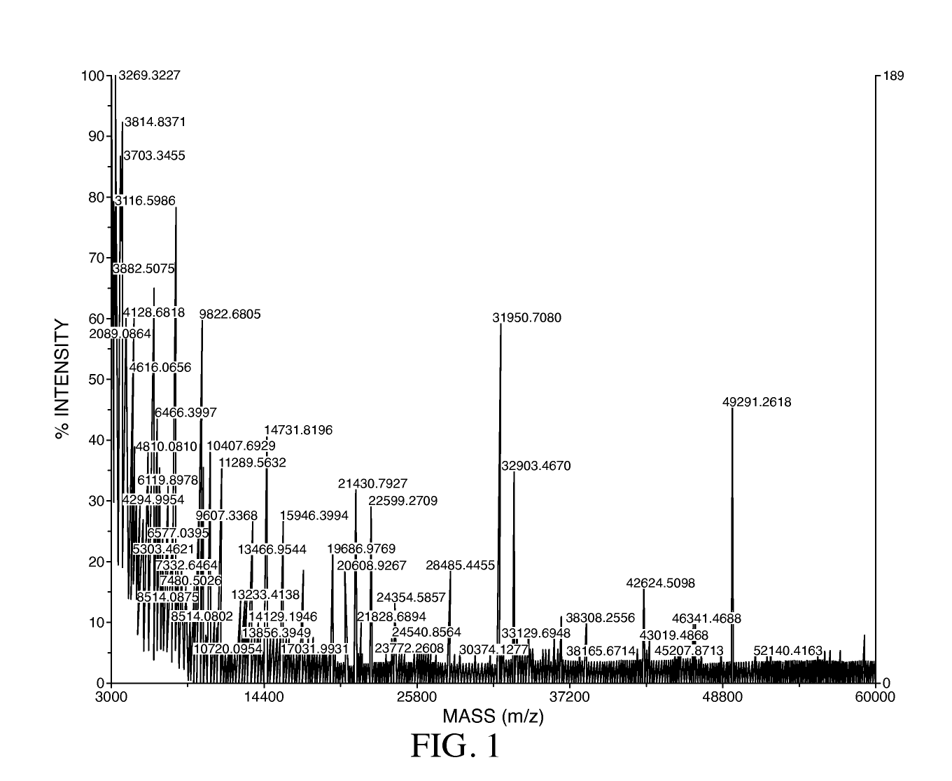

The conditions in which amyloid β may oligomerize in nervous tissue, a milieu of aqueous-soluble proteins with which it may associate, were attempted to be re-created to identify the more disease-relevant structural state of amyloid β oligomers and fibrils. Aqueous soluble proteins were prepared from rat brain by ultracentrifugation. Specifically, 5 volumes of TBS buffer (20 mM Tris-HCL, pH 7.5, 34 mM NaCl and a complete protease inhibitor cocktail (Santa Cruz) per gram of brain tissue was added to the rat brain tissue on ice b. Dounce homogenization was then carried out with a tight-fitting pestle. The homogenized brain tissues were then centrifuged at 150,000×g for 1 hour at 4° C. (40,000 rpm Ty65). The infranatant (between floating myelin and a half cm above the pellet) was then removed and aliquots were frozen at −75° C. The pellets were then resuspended in TBS to the original volume and frozen in aliquots at −75° C. Synthetic, monomeric human amyloid β 1-42 was added to this mixture to provide a final concentration of 1.5 uM amyloid β, and the solution was incubated for 24 hours at 4° C. Centrifugation of the mixture at 5,800 g for 10 minutes was performed to remove fibrillar assemblies and then Immunoprecipitation was performed using 6E10 conjugated agarose spin columns (Pierce Chemical Company) for 24 hours at 4° C. The eluted amyloid β oligomers were then subject to MALDI-Tof mass spectroscopic analysis to identify the contents of the sample, FIG. 1.

As illustrated in FIG. 1, the amyloid β self-associated in the protein containing solution to form subunit assemblies of 22,599 Da, 5 subunit pentamers and 31,950 Da, 7 subunit, 7 mers. Another peak at 49,291 Da may represent 12 subunit, 12 mers, although this would not appear to be an accurate molecular weight for amyloid β 12 mers. Notably, no peaks are observed at either 4518 Da or 9036 Da which would represent amyloid β monomers and dimers. However, peaks at 9,882 Da and 14,731 Da could represent amyloid β dimers associated with a 786 Da (or 2×393 Da) lipids or proteins and amyloid β trimers associated with 3×393 Da lipids or proteins, respectively. In addition, the presence of a peak at 19,686 Da is indicative of an assembly state possibly involving a trimer complex and a rat amyloid β fragment of 4954 Da. Accordingly these data may reflect the association of small lipids or proteins with dimers and trimers of amyloid β which may direct the assembly of conformational states unique to physiological systems.

### EXAMPLE 2

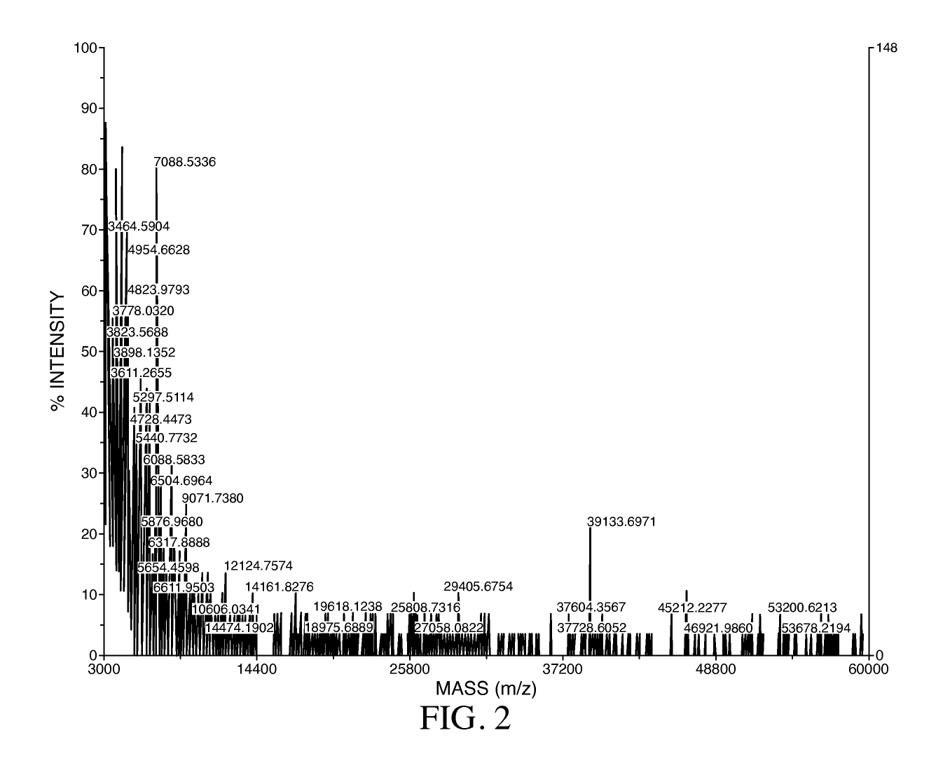

A solution of 1.5 uM monomeric human amyloid β 1-42 in a mixture of rat brain soluble proteins was incubated for 24 hours at 4° C. as described in Example 1. This solution was then treated with tri-fluoro ethanol (TFE) prior to taking the spectra. In TFE, assembled protein structures and non-covalently bound protein complexes dissociate into denatured proteins, and the peaks associated with assembled oligomers are expected to disappear. As shown in FIG. 2, the majority of protein peaks observed in FIG. 1 have disappeared including the 9822 Da, 14,731 Da, 31,950 Da, and 49,291 Da peaks identified above. However, an abundant peak is observed at 4518 Da which represents amyloid β monomer peak. A peak at 4954.7 is apparent which may represent a longer abeta fragment similar to amyloid β 1-46. An additional peak is observed at 7086 Da which is not present in FIG. 1, which may represent amyloid β monomers associated with a 2550 Da covalently bound protein.

### EXAMPLE 3

### Methods

TBS soluble extracts: Samples of post-mortem brain tissue from human patients characterized via histopathological analysis as Braak Stage V/VI Alzheimer's disease (AD) were obtained from Rhode Island Hospital brain tissue bank. Age and gender matched AD and normal tissue specimens were diluted to 0.15 gm tissue/ml in 20 mM Tris-HCL, 137 mM NaCl, pH 7.6 containing 1 mM EDTA and 1 mg/ml complete protease inhibitor cocktail (Sigma P8340) and homogenized. Ultracentrifugation of the tissue homogenates was performed at 105,000 g for 1 hour in a Beckman Optima XL-80K Ultracentrifuge. The resulting TBS soluble fractions were immunodepleted using protein-A and protein-G agarose columns (Pierce Chemical) and then size fractionated with Amicon Ultra 3, 10 & 100 kDa NMWCO filters (Millipore Corporation).

Immunoprecipitation: Size fractionated and immunodepleted TBS soluble extracts were concentrated to approximately 200 ul in the appropriate NMWCO Amicon Ultra filters. The concentrated TBS soluble extracts were diluted up to 400 ul with TBS sample buffer (Pierce Chemical) and centrifuged for 10 minutes at 5,800 g to remove fibrils. The resulting supernatant was then immunoprecipitated with 6E10-conjugated agarose beads overnight @4 C followed by antigen elution using high osmotic strength—Gentle elution buffers (Pierce Chemical) to isolate Abeta containing protein species.

MALDI-mass spectrometry: Immunoisolated beta amyloid was subjected to mass spectroscopic analysis using an Applied Biosystems (ABI) Voyager DE-Pro MALDI-Tof instrument. Samples were analyzed using various matrix types such as α-Cyano-4-hydroxycinnamic acid (CHCA), Sinapic acid (SA), or 6-Aza-2-thiothymine (ATT) depending on the target molecular weight range of the analysis. The instrument was run in a linear-positive ion mode along with a variable extraction delay. Non-accumulated spectra represented 100 shots of a “hot spot” per acquisition while accumulated spectra were represented by 12 separate areas of each spot with 200 laser shots per acquisition.

Data analysis: Data acquisition and analysis was performed using Voyager's Data Explorer software package. Standard processing of the mass spectra included smoothing of the spectrum and baseline subtraction functions in addition to variations in the signal to noise ratio.

ELISA for Ab quantification: Immunoprecipitated TBS soluble fractions were analyzed for both “total” Abeta and Abeta oligomer concentration using a modified sandwich ELISA technique. Briefly, 6E10 and 4G8 coated Nunc MaxiSorp 96-well plates were incubated with Abeta containing samples and then probed with a Biotinylated 4G8 detection antibody. Incubation with Streptavidin-HRP (Rockland) followed by development of a Tetramethyl benzidine (TMB) substrate allowed for colorimetric detection (OD 450) of abeta on a BioTEk Synergy HT plate reader. Monomeric Abeta 1-42 was used for generation of a standard curve and along with GEN 5 software allowed for quantification of Abeta levels in the immuno-precipitated samples.

1. A composition comprising: neural proteins suspended in an aqueous solution to form a neural protein slurry; and synthetic amyloid β, wherein the synthetic amyloid β is present at a concentration in the composition of about 0.5 μM to about 10 mM.

2. The composition of claim 1, wherein the neural proteins are brain proteins.

3. The composition of claim 1, wherein the neural proteins are derived from the brain of a mammal.

4. The composition of claim 1, wherein the amyloid β is selected from the group consisting of amyloid β monomers, amyloid β oligomers of about 2 to about 200 amyloid β monomers, or combinations thereof.

5. The composition of claim 1, wherein the amyloid β is selected from the group consisting of amyloid β monomers, amyloid β oligomers of 2 or 100 amyloid β monomers, and combinations thereof.

6. The composition of claim 1, wherein the protein complexes comprise amyloid β oligomers associated with one or more neural proteins from the neural protein slurry.

7. The composition of claim 1, wherein the protein complexes comprise amyloid β fibrils associated with one or more neural proteins from the neural protein slurry.

8. The compound of claim 1, wherein the aqueous solution further comprises one or more additional components selected from the group consisting of protease inhibitors, buffers, chelating agents, metals, metal salts, and combinations thereof.

9. The compound of claim 1, wherein the aqueous solution further comprises metal ions selected from the group consisting of Cu(ii), Zn(II), Fe(II), Fe(III), and Mg(II).

10-39. (canceled)

40. The composition of claim 1, wherein the amyloid β is selected from the group consisting of amyloid β oligomers of about 2 to about 200 amyloid β monomers, or combinations thereof.

41. The composition of claim 40 further comprising amyloid β monomers.

42. The composition of claim 1, wherein the amyloid β is selected from the group consisting of amyloid β oligomers of 2 or 100 amyloid β monomers, and combinations thereof.

43. The composition of claim 42 further comprising amyloid β monomers.

44. The composition of claim 1, wherein the synthetic amyloid β is amyloid β 1-42.
